(19) United States
(12) Patent Application Publication
(10) Pub. No.: US 2017/0269042 A1
(43) Pub. Date: Sep. 21, 2017

(54) SELECTIVE ISOLATION OF ARN ACIDS FROM CRUDE OILS

(71) Applicant: ExxonMobil Research and Engineering Company, Annandale, NJ (US)

(72) Inventors: Changyub Paek, Raritan, NJ (US); Anthony S. Mennito, Flemington, NJ (US)

(21) Appl. No.: 15/450,612

(22) Filed: Mar. 6, 2017

Related U.S. Application Data
(60) Provisional application No. 62/309,643, filed on Mar. 17, 2016.

(51) Int. Cl. G01N 30/96; B01D 15/36; B01J 47/00; B01J 41/04; C07C 51/47; C10G 29/20

(52) U.S. Cl. CPC G01N 30/96; C07C 51/47; C10G 29/20; B01J 47/00; B01J 41/04; B01D 15/361; C10G 2300/203; H01J 49/0027

(57) ABSTRACT

A process for selective isolation of high molecular weight (˜1230 Daltons) naphthenic acids (Arn acids). The process includes providing a polymeric resin with a bound a quaternary amino group and applying a crude oil sample containing Arn acids to the polymeric resin. A first wash of an organic solvent is applied to the sample followed by a second wash of a polar organic solvent mixture. The first two washes remove unwanted crude oil compositions while the Arn acids are bound to the quaternary amino groups. A third wash of acidified organic solvent removes the Arn acids from the polymeric resin, thereby forming an elute comprising the Arn acids and the acidified organic solvent. The acidified organic solve is then evaporated isolating the Arn acids from the crude oil sample.

### CROSS-REFERENCE TO RELATED APPLICATIONS

This application claims priority to U.S. Provisional Application No. 62/309,643 filed Mar. 17, 2016, which is herein incorporated by reference in its entirety.

### FIELD

Methods are provided for the selective isolation of Arn acids from crude oil samples.

### BACKGROUND

The presence of high molecular weight (˜1230 Daltons) naphthenic acids (Arn acids) can create a number of problems during the production, transportation, and separation of crude oils. For example, Arn acids are corrosive, favor the formation of emulsions, precipitate in the presence of calcium to form calcium naphthenate deposits, and can be detrimental to the environment. Such problems can result in loss of oil production through facility damage and unplanned operational shutdowns for cleanup.

Understanding the presence and composition of Arn acids in crude oil is useful for proper management strategies for mitigating the potential damage. Mass spectrometry techniques have been used to confirm the presence of Arn acids. The complex nature of crude oils, however, makes it difficult to analyze for specific compounds like Arn acids due to the numerous interferences typically present in crude oils. This difficulty is perhaps compounded by the fact that Am acids are present in crude oils at low concentrations such as 50 ppm or less.

The prior art, for the most part, describes extraction of Arn acids from naphthenate deposits. For example, Simon et al. describes a method for separation of Arn acids from a naphthenate deposit using a quaternary amine based ion exchange resin. See Simon et al., Determination of C₈₀ Tetra-acid Content in Calcium Naphthenate Deposits, 1200 J. OF CHROMATOGRAPHY 136-43 (2008). This is not particularly helpful in the current context because a naphthenate deposit already contains concentrated Arn acid. It would be more useful to be able to determine the presence and amount of Arn acid by directly testing the crude oil itself

U.S. Pat. No. 8,084,264 discloses separation of Arn acids directly from crude oil. The method involves contacting the crude oil sample with gaseous ammonia to form a reaction product. The reaction product is then aged at a reduced temperature for a period sufficient to form a precipitate of the reaction product. The reaction product is then analyzed via high resolution mass spectrometry to determine the presence of Arn acids. This method is undesirable because it requires use of hazardous ammonium gas and multiple apparatus with temperature control. It also takes days of operation to obtain final extracts.

U.S. Pat. No. 8,674,161 also discloses separation of Arn acids directly from crude oil. The method describes contacting the crude oil sample with a solid Arn absorption/adsorption medium, separating the solids from the remaining crude after the Arn acids have been absorbed by or adsorbed on the solids, washing the solids with an organic solvent, bringing the solids in contact with a mixture of acidified water or other acid and an organic solvent to release the Arn acids from the absorption/adsorption medium, and quantifying the Arn acids in the organic phase. The method further describes that the absorption/adsorption medium consists of hydroxides, oxides, carbonates, or bicarbonates of alkaline earth metals, alkali metals, or transition metals. The absorption/adsorption medium may also consist of other basic transition metal salts, silica, modified silica, or sephadex. The examples disclosed describe using 20 to 100 g of crude oil to carry out the method. This method is undesirable because it uses a large quantity of crude, which limits its broad application for diverse crude oils as their availability can be constrained. This method can also generate larger volume of waste.

There is a need for a simpler, more efficient process to determine Arn contact in crude oils. The currently disclosed process offers a quick and efficient means to extract low concentration Arn acids directly from crude oil samples. It does not involve any reaction, but rather only isolation and recovery processes at room temperature. Arn acids are selectively isolated from crude oils through ionic interaction with a strong basic group, such as a quaternary amino group, bound to polymeric resins and recovered through an ion-exchange mechanism. The Arn acids are then recovered by using an acidified organic solvent. The process requires only a small crude oil sample, as low as 3 mL, and generates minimal waste.

### SUMMARY

In various aspects, provided herein is a method to isolate high molecular weight naphthenic acids (Arn acids) in a crude oil sample, comprising: providing a polymeric resin; a quaternary amine bound to the polymeric resin; applying a crude oil sample containing Arn acids to the polymeric resin; applying a first wash to the polymeric resin with the applied crude oil sample, the first wash comprising an organic solvent; applying a second wash to the polymeric resin with the applied crude oil sample, the second wash comprising a polar organic solvent mixture; applying a third wash to the polymeric resin with the applied crude oil sample, the third wash comprising an acidified organic solvent, thereby forming an elute comprising the Arn acids and the acidified organic solvent; and evaporating the elute to remove the acidified organic solvent thereby isolating the Arn acids.

In another aspect, the organic solvent may comprise toluene and/or xylene. The polar organic solvent mixture may comprise toluene or a xylene and methanol, ethanol, or 2-propanol, such as a mixture of toluene and methanol. In another aspect, the polar organic solvent mixture is mixed at a molar ratio of 2:1 (toluene or xylene : methanol, ethanol, or 2-propanol), such as the toluene and methanol are mixed at a molar ratio of 2:1 (toluene:methanol). In yet another aspect, the acidified organic solvent can include formic acid in methylene chloride.

The method can be performed with a crude oil sample that is approximately 3 ml. In one aspect, the first wash comprises 6-15 ml of the organic solvent. In another aspect, evaporating the elute occurs under nitrogen. In another aspect, the method may include the additional act of analyzing the isolated Arn acids by mass spectrometry.

### BRIEF DESCRIPTION OF THE DRAWINGS

FIG. 1 illustrates a general diagram of the Arn acid extraction process disclosed herein.

FIG. 2 illustrates a mass spectrometry comparison of Arn acid distribution between crude oil samples across the world.

FIG. 3 illustrates a comparison of Arn acid concentration between a naphthenic deposit (top) measured by mass spectrometry with negative electrospray ionization and a crude oil sample from the same reserve measured by the process disclosed herein.

### DETAILED DESCRIPTION

In various aspects, methods for isolating and extracting Arn acids from crude oil samples are provided. In one or more aspects, a polymeric resin with a quaternary amine bound to the polymeric resin is loaded onto a cartridge, thereby creating an effective ion exchange resin. It should be noted that any ion exchange resin, or insoluble polymer matrix, with strong basic groups bound to its polymer would be an effective medium to perform the disclosed process. The polymeric resin is an insoluble matrix in the form of small beads fabricated from an organic polymer substrate. The beads are typically porous, providing a high surface area. The trapping of ions occurs with the accompanying releasing of other ions; thus the process is called ion-exchange. It would be recognized by a person of skill in the art that there are multiple types of polymeric resins that would be effective as an ion-exchange resin in the currently disclosed process. A crude oil sample is applied to the polymeric resin. The crude oil sample may contain a concentration of Arn acids. The Arn acids bond through electrostatic interaction with the strong basic groups, such as quaternary amines, bound to the polymeric resin.

In another aspect a first wash is applied to the polymeric resin with the applied crude oil sample. The first wash may comprise an organic solvent. Organic solvents are carbon-based solvents, such as toluene or xylene to dissolve non-polar hydrocarbon components in crude oil. The first wash serves to remove a majority of the crude oil while the Arn acids remain bound to the polymeric resin as their negatively charged acidic groups electrostatically interact to the basic groups with positive charges. The first wash may be applied until no colored species are eluted from the cartridge.

In various aspects a second wash may then be applied. The second wash may comprise a polar organic solvent mixture, such as a mixture of toluene or xylene with methanol, ethanol, or 2-propanol or mixtures thereof. The second wash serves to remove any unwanted crude oil components remaining after the first wash. The second wash removes more polar species within the crude oil sample, thereby minimizing potential interferences. It is necessary to perform two washing steps with solvents having different polarities because of the complex and diverse polarity of components in crude oil. While the first solvent tends to dissolve and remove the non-polar hydrocarbon components, the second wash serves to dissolve and remove the more polar hydrocarbon components. The second wash may be applied until no colored species are eluted from the cartridge.

In various aspects a third wash may then be applied. The third wash may comprise an acidified organic solvent. As used herein, the term acidified organic solvent refers to volatile organic solvents with dissolved organic acids. At this point in the process, preferably only Am acids remain bound to the polymeric resin. The acidified organic solvent wash serves to remove the Am acids from the polymeric resin forming an elute comprising the Am acids and the acidified organic solvent. The low pH of the acidified organic solvent tends to neutralize the Am acids, causing them to lose their electrostatic interactions with the quaternary amino groups on the polymeric resin. The elute can then be evaporated to remove the acidified organic solvent thereby isolating the Am acids.

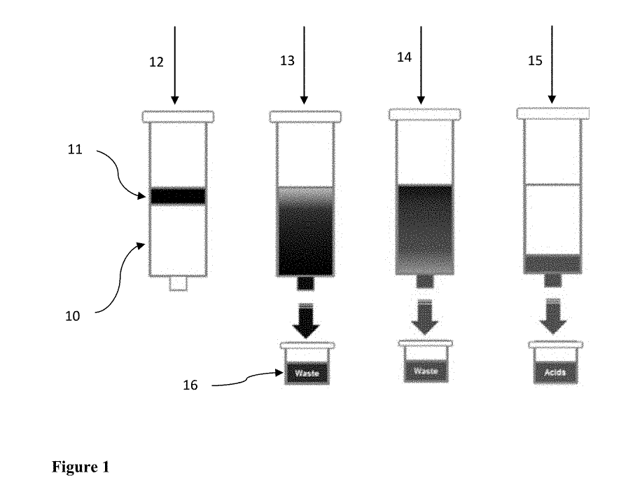

An embodiment of the process is described with reference to FIG. 1. Complex 11 comprises a cartridge loaded with a polymeric resin. A strong basic group, such as a quaternary amino group, is bound to the polymeric resin. Complex 11 is loaded within syringe 10. Crude oil sample 12, which comprises Am acids, is then loaded onto complex 11. Only a small amount of crude oil sample 12, about 3 mL, is needed to perform the process. First wash 13 is applied to crude oil sample 12. First wash 13 comprises an organic solvent. Unwanted crude oil components are eluted from syringe 10 into receptacle 16 as a result of the first wash 13. Second wash 14 is then applied to crude oil sample 12. Second wash 14 comprises a polar organic solvent mixture. The second wash 14 preferably removes any remaining unwanted crude oil components from complex 11. At this point, it is preferred that only Am acids from crude oil sample 12 remain bound to complex 11. A third wash 15 is then applied to complex 11. Third wash 15 comprises an acidified organic solvent and serves to remove the Am acids from complex 11, forming an elute which comprises Arn acids and the acidified organic solvent. The acidified organic solvent can then be evaporated thereby isolating the Arn acids. The Arn acids content of the original crude oil sample 12 can then be analyzed via mass spectrometry or any other viable analytical technique.

As will be shown in the examples below, the above described process provides a simpler, faster, and more efficient method to quantify Arn acid content within a crude oil sample than that which is disclosed in the prior art.

### Additional Embodiments

Embodiment 1: A method to isolate high molecular weight naphthenic acids (Arn acids) in a crude oil sample, comprising: providing a polymeric resin; a quaternary amine bound to the polymeric resin; applying a crude oil sample containing Arn acids to the polymeric resin;

applying a first wash to the polymeric resin with the applied crude oil sample, the first wash comprising an organic solvent; applying a second wash to the polymeric resin with the applied crude oil sample, the second wash comprising a polar organic solvent mixture; applying a third wash to the polymeric resin with the applied crude oil sample, the third wash comprising an acidified organic solvent, thereby forming an elute comprising the Arn acids and the acidified organic solvent; and evaporating the elute to remove the acidified organic solvent thereby isolating the Arn acids.

Embodiment 2: The method of embodiment 1, wherein the organic solvent comprises toluene.

Embodiment 3: The method of any of the previous embodiments, wherein the organic solvent comprises a xylene.

Embodiment 4: The method of any of the previous embodiments, wherein the polar organic solvent mixture comprises toluene or a xylene and methanol, ethanol, or 2-propanol.

Embodiment 5: The method of any of the previous embodiments, wherein the polar organic solvent mixture comprises toluene and methanol.

Embodiment 6: The method of any of the previous embodiments, wherein the crude oil sample is approximately 3 ml.

Embodiment 7: The method of any of the previous embodiments, wherein the first wash comprises 6-15 ml of the organic solvent.

Embodiment 8: The method of any of the previous embodiments, wherein the polar organic solvent mixture is mixed at a molar ratio of 2:1 (toluene or xylene:methanol, ethanol, or 2-propanol).

Embodiment 9: The method of any of the previous embodiments, wherein the toluene and methanol are mixed at a molar ratio of 2:1 (toluene:methanol).

Embodiment 10: The method of any of the previous embodiments, wherein the acidified organic solvent comprises formic acid in methylene chloride.

Embodiment 11: The method of any of the previous embodiments, wherein the evaporating the elute occurs under nitrogen.

Embodiment 12: The method of any of the previous embodiments, further comprising, analyzing the isolated Arn acids by mass spectrometry.

Although the present invention has been described in terms of specific embodiments, it is not so limited. Suitable alterations/modifications for operation under specific conditions should be apparent to those skilled in the art. It is therefore intended that the following claims be interpreted as covering all such alterations/modifications as fall within the true spirit/scope of the invention.

### EXAMPLES

### Example 1

### An Embodiment of the Disclosed Process

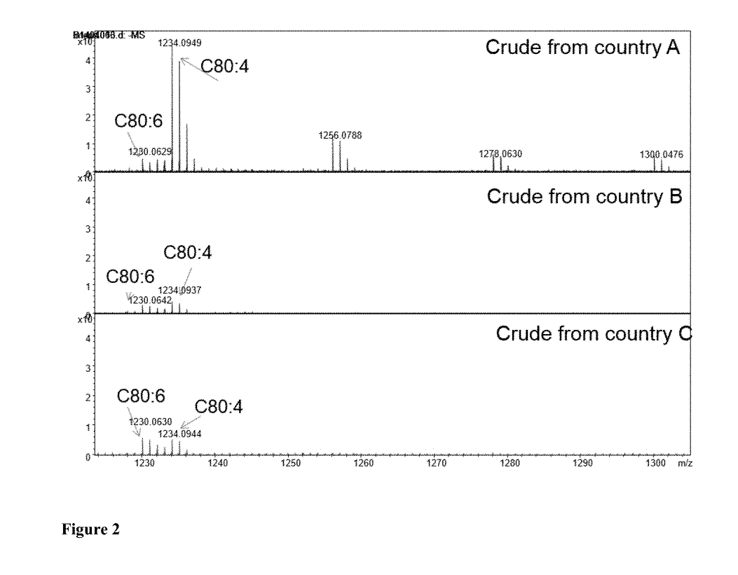

FIG. 2 illustrates a mass spectroscopy spectrum for Arn acid isolation from a crude oil samples using the process disclosed herein. The process was carried out using a commercial ion exchange resin, specifically a Bond Elute PAX cartidge (6 ml) containing 200 mg of porous resins with 90 umol/g capacity (Agilent), was used as separation media. It is quaternary amine bound to polymer resin that offers high stability when applying acidic solvents for ARN acids recovery. All procedures were conducted at room temperature. The resin was first washed with water followed by methanol and toluene. About 3 ml of crude is loaded on a cartridge, then the cartridge is washed with toluene (6-12 ml) until no colored species were eluted. A mixture of toluene and methanol (2:1 ratio) was then applied (3-5 ml) until no colored species were eluted (wash 2). These two washing steps remove most of oil and unwanted components. Finally, 1M formic acid in methylene chloride was applied to recover ARN acids from resins. Solvents were evaporated under nitrogen, and the extract was analyzed by mass spectrometry. FIG. 2 illustrates a comparison of Arn acid distribution between crude oil samples across the world. This figure shows that the process disclosed herein is effective for crude oil sample having different compositional makeup.

### Example 2

### Comparison between Naphthenate Deposit and Crude Oil in Same Oil Field

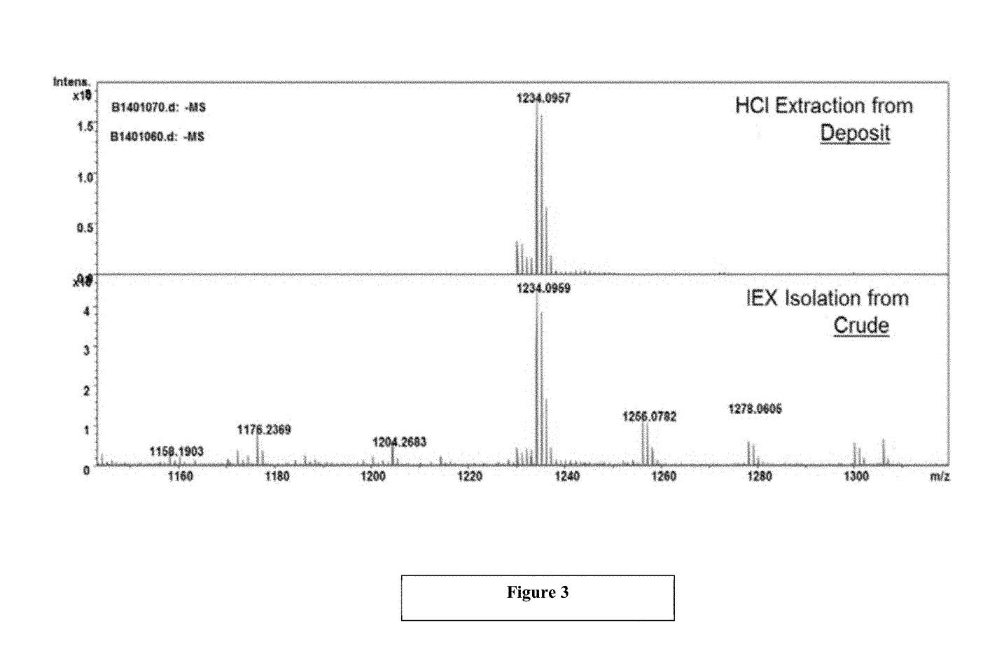

FIG. 3 illustrates a comparison of Arn acid distribution between a naphthenic deposit (top) measured by mass spectrometry after acid digestion of the deposit using toluene and hydrogen chloride and a crude oil sample (bottom) from the same oil field measured by the process disclosed herein. Using the method described in Example 1, it is shown that the disclosed process—performed on crude oil—provides accurate results when compared to prior methods used on naphthenic deposits, which contain a highly concentrated Arn acid content. In other words, this demonstrates that the current method accurately captures Arn acid composition of a crude oil sample as shown by agreement with the Arn acid concentration found the Arn enriched deposit.

### Example 3

### Repeatability of Process

The process disclosed herein was carried out three times on the same crude oil sample.

The results are displayed in Table 1.

TABLE 1

| | Arn acids |
|---|---|
| Crude X | (ppm, μg/ml) |
| Run 1 | 2.2 |
| Run 2 | 2.7 |
| Run 3 | 2.1 |
| Average/% RSD | 2.3/13% |

As shown, the disclosed method is precise, relative standard deviation of 13%, after repeating the process three times with the same crude oil sample.

1. A method to isolate high molecular weight naphthenic acids (Arn acids) in a crude oil sample, comprising:
providing a polymeric resin; a quaternary amine bound to the polymeric resin;
applying a crude oil sample containing Arn acids to the polymeric resin;
applying a first wash to the polymeric resin with the applied crude oil sample, the first wash comprising an organic solvent;
applying a second wash to the polymeric resin with the applied crude oil sample, the second wash comprising a polar organic solvent mixture;
applying a third wash to the polymeric resin with the applied crude oil sample, the third wash comprising an acidified organic solvent, thereby forming an elute comprising the Arn acids and the acidified organic solvent; and
evaporating the elute to remove the acidified organic solvent thereby isolating the Arn acids.

2. The method of claim 1, wherein the organic solvent comprises toluene.

3. The method of claim 1, wherein the organic solvent comprises a xylene.

4. The method of claim 1, wherein the polar organic solvent mixture comprises toluene or a xylene and methanol, ethanol, or 2-propanol.

5. The method of claim 4, wherein the polar organic solvent mixture comprises toluene and methanol.

6. The method of claim 1, wherein the crude oil sample is approximately 3 ml.

7. The method of claim 1, wherein the first wash comprises 6-15 ml of the organic solvent.

8. The method of claim 4, wherein the polar organic solvent mixture is mixed at a molar ratio of 2:1 (toluene or xylene:methanol, ethanol, or 2-propanol).

9. The method of claim 5, wherein the toluene and methanol are mixed at a molar ratio of 2:1 (toluene:methanol).

10. The method of claim 1, wherein the acidified organic solvent comprises formic acid in methylene chloride.

11. The method of claim 1, wherein the evaporating the elute occurs under nitrogen.

12. The method of claim 1, further comprising, analyzing the isolated Am acids by mass spectrometry.
